(19) United States
(12) Patent Application Publication
(10) Pub. No.: US 2018/0214631 A1
(43) Pub. Date: Aug. 2, 2018

(54) SMART CARTRIDGE SYSTEM FOR CONTAINING AND RELEASING MEDICAMENT WITH PUMPING MECHANISM AND COMPRESSIBLE RESERVOIR

(71) Applicant: Picolife Technologies, LLC, Jacksonville, FL (US)

(72) Inventor: Farid Amirouche, Highland Park, IL (US)

(21) Appl. No.: 15/423,573

(22) Filed: Feb. 2, 2017

(51) Int. Cl. A61M 5/142; A61M 5/168

(52) U.S. Cl. CPC A61M 5/14244; A61M 5/16809; A61M 2205/7536; A61M 2205/50; A61M 2205/3379

(57) ABSTRACT

The present disclosure relates to the field of treatment of symptoms or disorders through use of a cartridge system that may be used in conjunction with a delivery system, such as an infusion set. More specifically, the present disclosure describes a smart cartridge for containing and releasing medicament, wherein the smart cartridge may comprise a system that may be operable when in electrical communication with an external power source. A smart cartridge may comprise one or more compressible reservoirs that may contain the medicament, wherein the release of medicament from a compressible reservoir may be controlled by a pumping mechanism within the cartridge. In some aspects, a smart cartridge may be operable when paired or connected with a portable dispensing unit or other power source and control mechanism, wherein the pumping mechanism may individually actuate a compressible reservoir and deliver medicaments continuously or at a programmable intermittent rate.

### BACKGROUND OF THE DISCLOSURE

Diabetes is a complex disease caused by the body's failure to produce adequate insulin or the cell's failure to respond to insulin, resulting in high levels of glucose in the blood. Type I diabetes is a form of Diabetes Mellitus that results from autoimmune destruction of insulin-producing beta cells of the pancreas in genetically predisposed individuals. There is no current cure and treatment by injection or infusion of insulin must be continued indefinitely. Type II diabetes is a metabolic disorder brought on at any age and time by a combination of lifestyle, diet, obesity, and genetic factors. The World Health Organization recently revised its findings from a study conducted in 2004 with predictions that by 2030, 10% of the world's population of all ages will have either Type I or Type II diabetes. This translates to roughly 552 million people worldwide suffering from some form of this disease.

Typically, treatment for diabetes requires both repeated checking of blood glucose levels and several injections of insulin as prescribed by a physician throughout the day since insulin cannot be taken orally. Major drawbacks of such treatment are the constant need to draw blood and test glucose levels throughout the day, administering improper or low dosage amounts of insulin, contamination of the insulin delivery system, lifestyle or financial restrictions, the unfortunate potential development of subcutaneous scar tissue due to repeated injections at the same location, and the high cost of medication, testing strips, and other treatment related materials.

Diabetes is usually controlled by insulin replacement therapy in which insulin is delivered to the diabetic person by injection to counteract elevated blood glucose levels. Recent therapies include the basal/bolus method of treatment in which the basal, a long acting insulin medication, such as, for example, Humalog® and Apidra®, is delivered via injection once every day. The basal provides the body with an insulin profile that is relatively constant throughout the day, or could follow a profile best suited for the diabetic person. These rates can change based on the patient's response to insulin. At meal-time, an additional dose of insulin, or bolus, is administered based on the amount of carbohydrates and protein in the meal. The bolus is viewed as an emergency response to spikes in blood sugar that need to be brought down by injection of insulin. Accurate calculations of various parameters, including the amount of carbohydrates and proteins consumed and the lapse in time since the last dosage are necessary to determine the appropriate dosage of insulin. As a result, the dosages are prone to human error and the method is ineffective when doses are skipped, forgotten, or miscalculated. Exercise, stress, and other factors can also cause the calculations to be inaccurate. Bolus is usually administered when the patient's glucose level is high or above certain acceptable thresholds and requires immediate attention.

To address these problems, insulin delivery devices or pumps were developed to attempt to mimic the way a normal, healthy pancreas delivers insulin to the body. Innovations are rapidly advancing toward the creation of a closed-loop insulin delivery system. These systems employ real-time glucose-responsive insulin administration via continuous glucose monitoring and wireless communication with a controller which dispenses insulin based on tightly controlled algorithms. The two main algorithmic systems used to calculate insulin dosages automatically are the proportional-integral-derivative (PID) control, or and the mathematic-predictive control (MPC). MPC algorithms can be considered proactive or predictive. They forecast glucose levels in anticipation of meals, physical activity and administer insulin over a prediction window of 1.5 to 3 hours or longer. PID algorithms are considered reactive in response to measured glucose levels and cannot predict dosages. Unfortunately, there is currently no industry-wide standard in place for embedded algorithmic calculations, and dose calculations vary from device to device.

Often, both methods are utilized when insulin is co-administered with glucagon or other medication, though silico simulations, or computer simulated, glycemic regulation via MPC calculations achieves superior glucose regulation.

Most insulin pumps today are programmed to deliver a continual basal dose of insulin and occasionally a bolus dose, usually performed manually, in response to a patient's meal intake and physical activities. Early pumps had many limitations which made them inconvenient and less effective. Their overall size, propensity to leak, and extremely high cost made them unusable for long-term disease management and financially out of reach for most patients with limited or no insurance coverage. These types of pumps were also potentially risky in terms of unintentionally over or under dosing a patient, because the accuracy of the dose administered is dependent upon the reliability of the piston-driven motor, and medication is delivered in quick bursts rather than diffused over time.

Conventional insulin pumps are worn outside the body and are connected to the user via a cannula that is inserted somewhere on the user's abdomen. The insulin is delivered under the skin and is absorbed into the body through the subcutaneous fat layer. Subcutaneous delivery of insulin takes advantage of the lack of blood flow in this area which allows for slower absorption of the medication through the dermal capillaries. Other methods of non-invasive insulin delivery have been explored and compared in Various Non-Injectable Delivery Systems for the Treatment of Diabetes Mellitus, Yadav, N., Morris, G., Harding, S., Ang, S., Adams, G., Endocrine, Metabolic& Immune Disorders-Drug Targets, 2009, Vol. 9 (1):1-13. The pump is worn on the user's body at all times, concealed by clothing as desired, and therefore should be as small and unobtrusive as possible. The tubing connecting the pump to the user must be relatively short as crystallization of the insulin medication is of great concern when the tubing is long.

One recurring problem with most miniaturized ambulatory infusion pumps available today is that the amount of medication which can be stored in the reservoirs often cannot meet the needs of certain diabetic patients. Many Type II diabetics who require insulin often need more insulin per gram of carbohydrate due to a condition referred to as “insulin resistance.” Additionally, many diabetic therapies include one or more medications delivered alternately or simultaneously. For this reason, a medication pump which employs a plurality of reservoirs able to dispense medication at variable rates is optimal. Therefore, a substantial need exists to best maximize the volume of the medication reservoirs while maintaining a very small overall size of the device itself.

With the demand for a decrease in size of the pump unit also comes a decreased size in the medication reservoir. This reduced reservoir size means more frequent refilling, greater potential for contamination of the reservoir, more frequent changes of the cannula and tubing, and greater expense overall in treating the condition. Frequent manual refilling of a medication reservoir can also lead to the increased formation of bubbles, which is a significant problem. Even very small bubbles of 10 microliters or less can displace enough fluid to equal a missed dose of 1 unit of medicament. Insulin medication itself can also form bubbles when dissolved air is “outgassed” through normal changes in temperature or atmospheric pressure. Therefore the need exists to provide a disposable, pre-pressurized, pre-filled medication reservoir that can work as part of a medication pump system to provide extremely accurate delivery of a plurality of medications.

### SUMMARY OF THE DISCLOSURE

What is needed is a smart cartridge that addresses the concerns laid out above while delivering an insulin treatment protocol that delivers on a variety of factors. The future of the insulin treatment protocol detailed above is vitally dependent upon several factors: more accurate glucose sensors, rapid response software and hardware, single catheters for both glucose sensing and medication diffusion, and dual or multi-chambered medication delivery cartridge systems. The present invention meets these current and future needs.

The present disclosure relates to the field of treatment of symptoms or disorders through use of a cartridge system that may be used in conjunction with a delivery system, such as an infusion set. More specifically, the present disclosure describes a smart cartridge for containing and releasing medicament, wherein the smart cartridge may comprise a system that may be operable when in electrical communication with an external power source.

One general aspect may include a smart cartridge, including: a first compressible reservoir comprising a first fill port configured to accept a filling mechanism, where the filling mechanism is configured to add a first medicament to the first compressible reservoir; a first overflow port configured to dispel an excess amount of the first medicament where the first medicament filled exceeds a first threshold volume capacity within the first compressible reservoir; a first flow port through which the first medicament flows for use; a first flexible pouch configured to contain the first medicament.

In some aspects, the smart cartridge may also include a first internal tubing connected to the first flow port. The smart cartridge may also include a pumping mechanism operably connected to the first internal tubing and configured to be electrically connected to an external power source, where the pumping mechanism controls flow of the first medicament from the first compressible reservoir. The smart cartridge may also include a flow rate sensor configured to monitor a flow of the first medicament from the first internal tubing to the first outlet tube. The smart cartridge may also include a first outlet tube connected to the first internal tubing through which a first expulsion of the first medicament to an external body flows. The smart cartridge may also include a housing containing the first compressible reservoir, the first internal tubing, the pumping mechanism, the flow rate sensor, and at least part of the first outlet tube.

Other embodiments of this aspect include corresponding components, systems, apparatus, and programming recorded on one or both internal or external storage devices, each configured to perform in concert with the functionality according to some embodiments of the present disclosure. Implementations may include one or more of the following features.

In some embodiments, the housing may comprise gas permeable through ports. The first compressible reservoir may further include a first end cap with the first fill port, the first overflow port, and the first flow port. The smart cartridge may further include a first volume sensor configured to recognize a volume level of the first medicament contained within the first compressible reservoir. In some aspects, the first compressible reservoir may further include: a first end cap with the first fill port and the first overflow port; and a second end cap with the first flow port. The first flow port may include a first internal tubing fitting mechanism that secures a connection between the first internal tubing and the first flow port.

In some implementations, the first compressible reservoir may further comprise: a first conductive layer adhered to a first wall of the first flexible pouch; and a second conductive layer adhered to a second wall of the first flexible pouch proximate to the second wall, where a predefined distance range between the first conductive layer and the second conductive layer is associated with a predefined capacitance range, and where each capacitance within the predefined capacitance range is associated with a first quantity of the first medicament contained in the first compressible reservoir.

In some aspects, the smart cartridge may further comprise a second compressible reservoir including: a second fill port configured to accept a second filling mechanism, where the second filling mechanism is configured to add a second medicament to the second compressible reservoir; a second overflow port configured to dispel an excess amount of the second medicament where the second medicament filled exceeds a second threshold volume capacity within the second compressible reservoir; a second flow port through which the second medicament flows for use; and a second flexible pouch configured to contain the second medicament. In some embodiments, a second expulsion of the second medicament from the second compressible reservoir may occur through the first outlet tube.

In some implementations, the pumping mechanism may alternately draw from the first compressible reservoir and the second compressible reservoir. The pumping mechanism may control flow of the first medicament from the first compressible reservoir until the first compressible reservoir reaches a minimum medicament volume threshold before controlling flow from the second compressible reservoir. The smart cartridge may further include a second outlet tube, where a second expulsion of the second medicament from the second compressible reservoir occurs through the second outlet tube. In some aspects, the pumping mechanism may alternately draw from the first compressible reservoir and the second compressible reservoir. In some embodiments, the pumping mechanism may control flow of the first medicament from the first compressible reservoir until the first compressible reservoir reaches a minimum medicament volume threshold before controlling flow from the second compressible reservoir.

In some implementations, the first medicament and the second medicament may be the same. In some aspects, the first medicament and the second medicament may be different. The the first threshold volume capacity and the second threshold volume capacity may be the same or different. In some aspects, the first compressible reservoir may be refillable. In some embodiments, the smart cartridge may be disposable and configured to be functional for one fill of the first medicament.

In the following sections, detailed descriptions of examples and methods of the disclosure will be given. The description of both preferred and alternative examples though thorough are exemplary only, and it is understood that to those skilled in the art variations, modifications, and alterations may be apparent. It is therefore to be understood that the examples do not limit the broadness of the aspects of the underlying disclosure as defined by the claims.

### BRIEF DESCRIPTION OF THE DRAWINGS

The accompanying drawings that are incorporated in and constitute a part of this specification illustrate several embodiments of the disclosure and, together with the description, serve to explain the principles of the disclosure:

FIG. 1A illustrates a perspective view of an exemplary smart cartridge with two compressible reservoirs and a single outlet, in accordance with some embodiments of the present disclosure.

FIG. 1B illustrates a perspective view of an exemplary smart cartridge with two compressible reservoirs and a single outlet, in accordance with some embodiments of the present disclosure.

FIG. 1C illustrates a perspective view of an exemplary smart cartridge with two compressible reservoirs and a single outlet, in accordance with some embodiments of the present disclosure.

FIG. 2A illustrates a perspective view of an exemplary smart cartridge with two compressible reservoirs and a single outlet tube with lower housing, in accordance with some embodiments of the present disclosure.

FIG. 2B illustrates a top view of an exemplary smart cartridge with two compressible reservoirs and a single outlet tube with lower housing, in accordance with some embodiments of the present disclosure.

FIG. 3 illustrates an exploded view of an exemplary smart cartridge with two compressible reservoirs and a single outlet, in accordance with some embodiments of the present disclosure.

FIG. 4 illustrates a perspective view of an alternate exemplary smart cartridge with two compressible reservoirs and two Luer-Lock outlets, in accordance with some embodiments of the present disclosure.

FIG. 5 illustrates a top down view of an alternate exemplary smart cartridge with two compressible reservoirs and two Luer-Lock outlets with the lower housing, in accordance with some embodiments of the present disclosure.

FIG. 6 illustrates an exploded view of an alternate exemplary smart cartridge with two compressible reservoirs and two dual Luer-Lock outlets, in accordance with some embodiments of the present disclosure.

FIG. 7A illustrates a perspective view of an alternate exemplary embodiment of a smart cartridge with lower housing, wherein the smart cartridge comprises reservoirs with different volume capacities, in accordance with some embodiments of the present disclosure.

FIG. 7B illustrates a top down view of an alternate exemplary embodiment of a smart cartridge with lower housing, wherein the smart cartridge comprises reservoirs with different volume capacities, in accordance with some embodiments of the present disclosure.

FIG. 8 illustrates a perspective view of an alternate exemplary embodiment of a smart cartridge with upper housing and lower housing, wherein the smart cartridge comprises reservoirs with different volume capacities, in accordance with some embodiments of the present disclosure.

FIG. 9A illustrates a perspective view of an exemplary embodiment of a compressible reservoir for use within a dual-reservoir smart cartridge, in accordance with some embodiments of the present disclosure.

FIG. 9B illustrates a perspective view of an exemplary embodiment of a compressible reservoir for use within a dual-reservoir smart cartridge, in accordance with some embodiments of the present disclosure.

FIG. 9C illustrates an exploded view of an exemplary embodiment of a compressible reservoir for use within a dual-reservoir smart cartridge, in accordance with some embodiments of the present disclosure.

FIG. 10A illustrates a perspective view of an end cap for a compressible reservoir, wherein the end cap may comprise a fill port with fill guide and an overflow port, in accordance with some embodiments of the present disclosure.

FIG. 10B illustrates a perspective view of an end cap for a compressible reservoir, wherein the end cap may comprise a fill port with fill guide and an overflow port, in accordance with some embodiments of the present disclosure.

FIG. 11A illustrates a perspective view of an end cap for a compressible reservoir, wherein the end cap may comprise an angled channel and a draw opening to which flexible tubing may be attached, in accordance with some embodiments of the present disclosure.

FIG. 11B illustrates a perspective view of an end cap for a compressible reservoir, wherein the end cap may comprise an angled channel and a draw opening to which flexible tubing may be attached, in accordance with some embodiments of the present disclosure.

FIG. 12 illustrates a cross section of an exemplary embodiment of a fill port for use with an end cap of a compressible reservoir of smart cartridge, in accordance with some embodiments of the present disclosure.

FIG. 13 illustrates a cross section of an exemplary open embodiment of an overflow port for use with a smart cartridge, wherein the overflow port may comprise an overflow to membrane and a through hole, in accordance with some embodiments of the present disclosure.

FIG. 14 illustrates a perspective view of an exemplary smart cartridge with a single compressible reservoir, in accordance with some embodiments of the present disclosure.

FIG. 15A illustrates a perspective view of an exemplary embodiment of a smart cartridge with a single compressible reservoir, in accordance with some embodiments of the present disclosure.

FIG. 15B illustrates a top down view of an exemplary embodiment of a smart cartridge with a single compressible reservoir, in accordance with some embodiments of the present disclosure.

FIG. 16 illustrates an exemplary embodiment of a smart cartridge, wherein the small compressible reservoir has a lower threshold volume capacity, in accordance with some embodiments of the present disclosure.

FIG. 17A illustrates a perspective view of a single compressible reservoir, wherein the single compressible reservoir comprises a flexible pouch, a first end cap with flow port, and a second end cap with fill port and overflow port, in accordance with some embodiments of the present disclosure.

FIG. 17B illustrates a perspective view of a single compressible reservoir, wherein the single compressible reservoir comprises a flexible pouch, a first end cap with flow port, and a second end cap with fill port and overflow port, in accordance with some embodiments of the present disclosure.

FIG. 18A illustrates an alternate exemplary embodiment of a smart cartridge with a single compressible reservoir, wherein the single compressible reservoir comprises a dual port end cap, in accordance with some embodiments of the present disclosure.

FIG. 18B illustrates an alternate exemplary embodiment of a smart cartridge with a single compressible reservoir, wherein the single compressible reservoir comprises a dual port end cap, in accordance with some embodiments of the present disclosure.

FIG. 19A illustrates an exemplary fill fitting, wherein the fill fitting may be used in conjunction with a compressible reservoir of a smart cartridge, in accordance with some embodiments of the present disclosure.

FIG. 19B illustrates an exemplary dual port end cap, wherein the dual port may be used in conjunction with a compressible reservoir of a smart cartridge, in accordance with some embodiments of the present disclosure.

FIG. 20A illustrates an exemplary embodiment of a pumping mechanism engaged with an external control unit, wherein control unit electromagnets may control the stroke of pump magnets interacting through a membrane, in accordance with some embodiments of the present disclosure.

FIG. 20B illustrates an exemplary embodiment of a pumping mechanism engaged with an external control unit, wherein control unit electromagnets may control the stroke of pump magnets interacting through a membrane, in accordance with some embodiments of the present disclosure.

FIG. 21A illustrates an exemplary embodiment of a flow rate sensor, wherein the flow rate sensor may detect flow of a medicament from a compressible reservoir through an outlet tube, in accordance with some embodiments of the present disclosure.

FIG. 21B illustrates an exemplary embodiment of a flow rate sensor, wherein the flow rate sensor may detect flow of a medicament from a compressible reservoir through an outlet tube, in accordance with some embodiments of the present disclosure.

FIG. 22 illustrates exemplary embodiments of wireless circuit boards of a pump display unit (PDU) and a smart cartridge, wherein the smart cartridge may be inserted into the PDU allowing for electrical communication between the smart cartridge and the PDU, in accordance with some embodiments of the present disclosure.

### DETAILED DESCRIPTION

The present disclosure relates to the field of a smart cartridge with dual reservoirs with an integrated pump actuation mechanism and collapsible capacitance controlled reservoirs. The smart cartridge includes a disposable pump with refill options on independent ports for delivery and control of more than one medicament such as insulin, glucagon, or a combination of therapeutic agents for the treatment management of type 1 and type 2 diabetic patients. More particularly, the disclosure relates to dual pump sensory cartridge pump devices with a microcontroller, feedback control, and self-monitoring of fluidic delivery. The current invention relates to the cartridge system with active control valves, along with volumetric flow sensors integrated into a dual chamber pump for storing and delivering medicament or other therapeutic agents for the treatment and management of ailments, such as, for example, diabetes or chronic pain.

The present disclosure relates to improving the use of medicament pumps to transport medicaments from a compressible reservoir to a patient such as through an infusion set, for delivery of insulin or other medicaments to a patient. More particularly, the disclosure relates to a smart cartridge of a medicament pump where the medicament reservoir and pump mechanism are combined into a single, cost-effective unit. In some embodiments, the pump cartridge unit may be a single-use disposable component configured to interact with a reusable pump or medicament distribution system. In some exemplary embodiments, the pump cartridge unit may be configured to prevent repeated uses, thereby ensuring the pump cartridge is disposable. In other embodiments, reservoirs with the pump cartridge unit may be refillable.

In the following sections, detailed descriptions of examples and methods of the disclosure will be given. The description of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

### Glossary

Smart Cartridge: as used herein refers to a system for containing and releasing medicament, wherein the system may be operable when in electrical communication with an external power source. In some embodiments, a cartridge may comprise one or more compressible reservoirs that may contain the medicament, wherein the release of medicament from a compressible reservoir may be controlled by a pumping mechanism within the cartridge. In some aspects, a cartridge may be operable when paired or connected with a portable dispensing unit (sometimes referred to as a “PDU”) or other device or system comprising a power source and control mechanism, such as a graphical user interface (sometimes referred to as a “GUI”), wherein the pumping mechanism may individually actuate a compressible reservoir and deliver medicaments continuously or at a programmable intermittent rate. As used herein, the term smart cartridge may be contrasted with a common use of the term cartridge traditionally used in medicament pumps, which typically comprises a passive container of the medicament.
Compressible Reservoir: as used herein refers to a partially flexible reservoir that may compress to the volume of medicament contained, wherein drawing medicament may compact adjacent walls of the flexible portion of the compressible reservoir and adding medicament may enlarge the interior of the flexible portion of the compressible reservoir. As used herein, a compressible reservoir may be contrasted with a traditional cartridge, which is typically rigid, and more recent soft reservoir embodiments, which may inflate and deflate like a balloon or bladder.

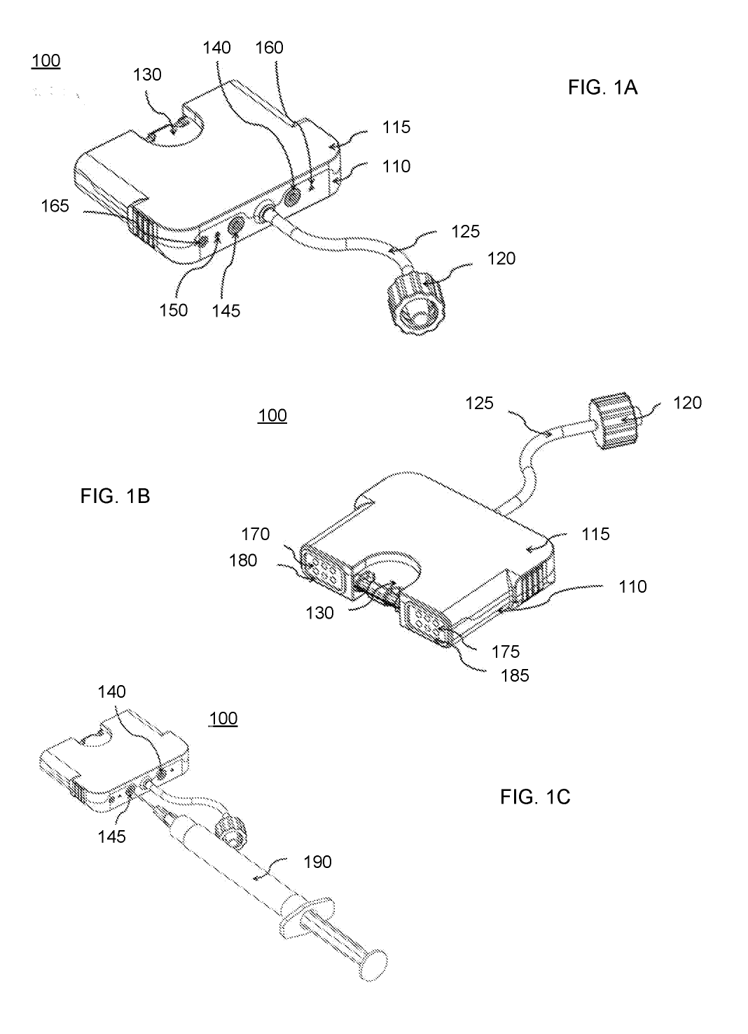

Referring now to FIGS. 1A-1C, perspective views of an exemplary smart cartridge 100 is illustrated. In some aspects, the smart cartridge 100 may comprise a first case part 110 and a second case part 115, wherein connecting, fitting, or combining the first case part 110 and the second case part 115 may create a cavity, which may house and contain one or more of the components of the smart cartridge 100. In some embodiments, the first case part 110 and the second case part 115 may comprise a substantially square or rectangular configuration. In some aspects, the first case part 110 and the second case part 115 may be permanently bonded, such as through glue, welding, magnets, slotted inserts, or other adhesive means.

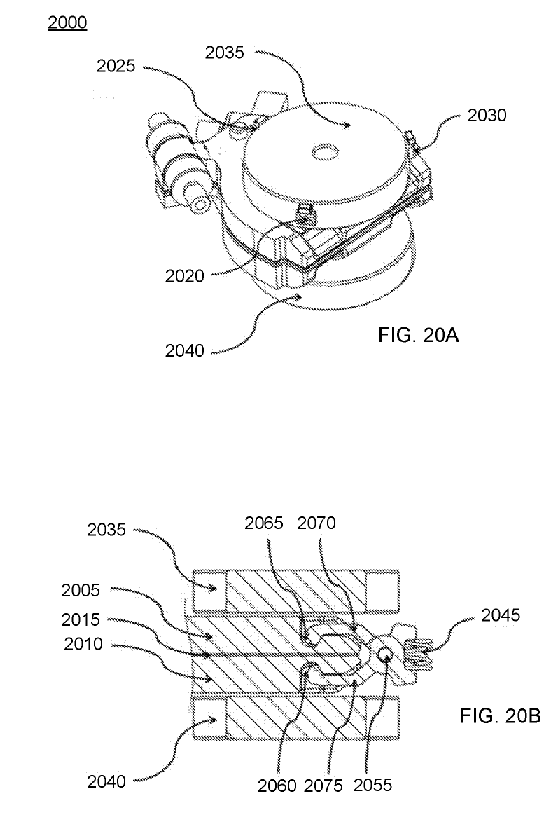

In some implementations, the smart cartridge 100 may comprise a pump mechanism 130 that may release medicament through a single outlet tube 125 with a Luer-Lock male fitting 120, which may be attached to a female fitting on an infusion set (not pictured). The smart cartridge 100 may further comprise a plurality of target connectors 170, 175 with a plurality of sealing gaskets 180, 185, as seen in FIG. 1B, which may secure placement within a pump display unit (not shown). The target connectors 170, 175 and sealing gaskets 180, 185 may ensure alignment and connection with electrical connectors and spring-loaded pins of a pump display unit, such as illustrated in FIG. 20.

In some aspects, the smart cartridge 100 may comprise refill ports 140, 145, wherein a syringe 190 may be inserted through the refill ports 140, 145 to inject medicament into the compressible reservoirs, as seen in FIG. 1C. In some implementations, overflow holes 160, 150 may provide a safety system to reduce overpressure, particularly during a refill. One or both the first case part 110 and the second case part 115 may comprise a plurality of through holes 165, which may be covered in the inside of the cartridge by a breathing membrane configured to allow passage of substances with predefined characteristics, for example, certain fluids, such as air or other gases, into and out of the smart cartridge 100, which may facilitate deflation or collapsing of reservoirs.

In some aspects, reservoirs may be filled or refilled through refill ports 140, 145 present in the first case part 110 with the aid of any suitable device, such as a syringe 190 configured to fluidly connect to the refill ports, such as shown in FIG. 1C. The refill ports 140, 145 may accommodate a variety of syringe types of diverse needle length and diameter, and may be easily distinguishable by the user to ensure refill of the compressible reservoir units with the correct drugs. For example, there may be distinctive shapes, design or colors, and may be marked with the name or type of drug to be used).

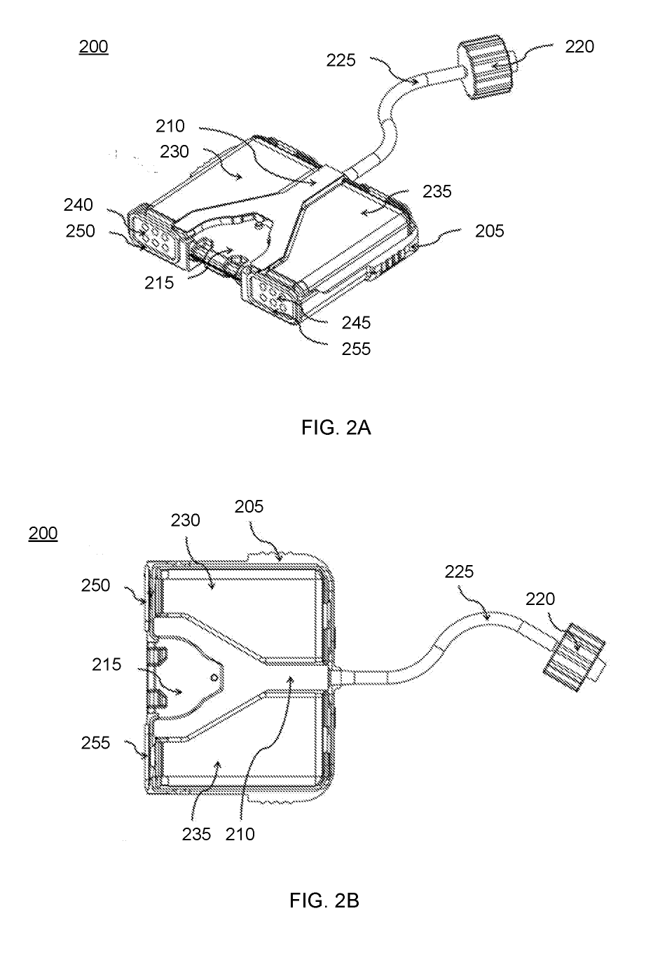

Referring now to FIGS. 2A-2B, a perspective view and a top view of an exemplary smart cartridge 200 with two reservoirs 230, 235 and a single outlet tube 225 is illustrated with lower housing 205. In some aspects, the single outlet tube 225 with Luer-Lock male fitting 220 may allow for the simultaneous release of fluid from the two reservoirs 230, 235. In some aspects, the two reservoirs 230, 235 may contain different fluids. For example, each reservoir 230, 235 may contain different medicaments, which may treat the same or different diseases, disorders, or symptoms. As another example, each reservoir 230, 235 may contain components of a medicament, wherein the simultaneous release may allow the components to mix to activate the medicament.

In some implementations, the pumping mechanism 215 may draw from the first compressible reservoir 230 and the second compressible reservoir 235 alternatingly. In some embodiments, the pumping mechanism 215 may draw from the first compressible reservoir 230 until the medicament level in the first compressible reservoir 230 reaches a threshold minimum volume, which may indicate the first compressible reservoir 230 no longer has medicament to draw from, wherein the pumping mechanism may then draw from the second compressible reservoir 235.

In some embodiments, each reservoir 230, 235 may be independently actuated by a pumping mechanism 215 that may cause an alternating release of medicament from the compressible reservoirs 230, 235. As illustrated, the compressible reservoirs 230, 235 may comprise the same shape and size, wherein each reservoir 230, 235 may have the same or similar volume capacities. In some embodiments, the internal infrastructure 210, which may house a flow rate sensor, such as illustrated and described in FIGS. 19A-19B, and at least part of the outlet tube 225. In some aspects, the smart cartridge 200 may comprise target connectors 240, 245 and sealing gaskets 250, 255, which may ensure alignment and connection with electrical connectors and spring-loaded pins of a pump display unit, such as illustrated in FIG. 20.

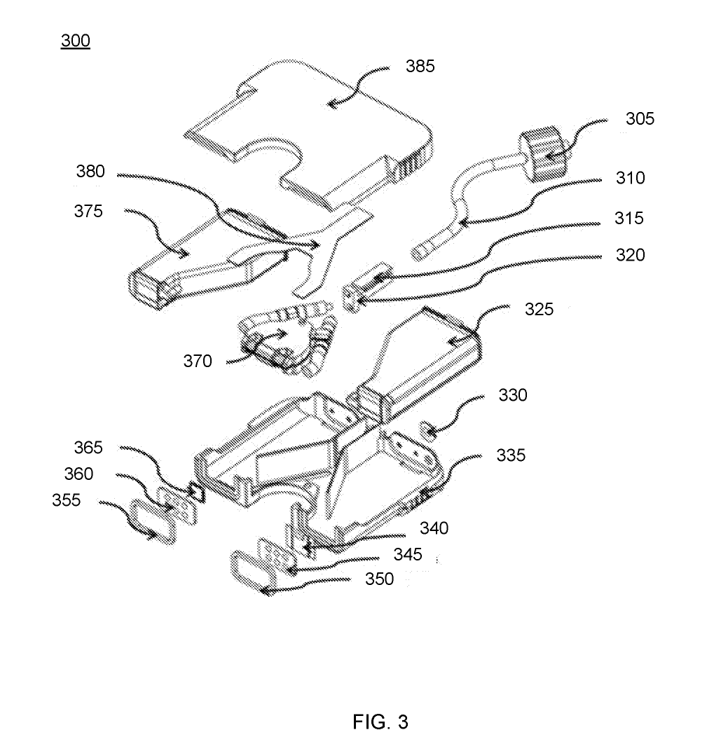

Referring now to FIG. 3, an exploded view of a smart cartridge 300 is illustrated. As illustrated in FIGS. 1A-2B, the smart cartridge 300 may comprise an upper housing 385, a lower housing 335, and an internal infrastructure 380, which may secure and organize components of the smart cartridge 300 within one or both the upper housing 385 and the lower housing 335. In some aspects, the upper housing 385 and the lower housing 335 may secure perimeter features and components, such as target connectors 360, 345, sealing gaskets 355, 350, and a breathing membrane 330, which may limit gas or moisture buildup within the smart cartridge 300.

In some embodiments, the smart cartridge 300 may comprise one or more thermal shut-off fuses 340, which may act as a quality control device. For example, to remain effective and safe, insulin must be maintained between 36° F. and 86° F. Outside of this threshold range, the desirable quality or properties of insulin may be adversely affected. In some aspects, each thermal shut-off fuse 340 may function differently, wherein a plurality of fail-safe devices limit the chance of dispensing potentially damaged medicament.

For example, one thermal shut-off fuse 340 may comprise a low temperature, melting alloy, which may melt if the internal environment of the smart cartridge 300 exceeds an acceptable threshold value. A second thermal shut-off fuse 340 may comprise a high thermal expansion liquid material, which may break its encapsulation and disable an electrical connection between the smart cartridge 300 and a power and control unit if the internal environment of the smart cartridge 300 drops below an acceptable minimum threshold temperature. In some embodiments, passive sensing mechanisms, such as the thermal shut-off fuse 340, may be set during the manufacturing process, which may allow the thermal shut-off fuse 340 to function throughout the life of the smart cartridge 300, indicating to a user if the temperature within the smart cartridge 300 ever fell out of the acceptable threshold temperature range.

In some implementations, the smart cartridge 300 may comprise a microcontroller 365, which may transmit data to an external device, such as a pump display unit (such as illustrated in FIG. 20), and may control at least a portion of the functionality of the smart cartridge 300.

In some aspects, the pumping mechanism 370 may direct the flow of medicament from a compressible reservoir 375, 325 through a T-connector 320. In some embodiments, the flows from each reservoir 375, 325 may merge inside the T-connector 320, and the resulting flow may be further directed through a flow rate sensor 315, and further through an outlet tube 310 and a male Luer-Lock fitting 305. The male Luer-Lock fitting 305 of the smart cartridge may be connected to any suitable delivery mechanism, including, for example, an infusion set, which may direct the medicament from the pump to the patient's subcutaneous tissue. In some aspects, components, such as the active valves surrounding the pumping mechanism 370, T-connector 320, flow rate sensor 315, and at least a portion of the outlet tube 310 may be organized and secured by the internal infrastructure. As an illustrative example, the internal infrastructure 380 may comprise guide walls to arrange components and coverings to protect components, such as a thin plastic sheet to cover tubing and the flow rate sensor 315.

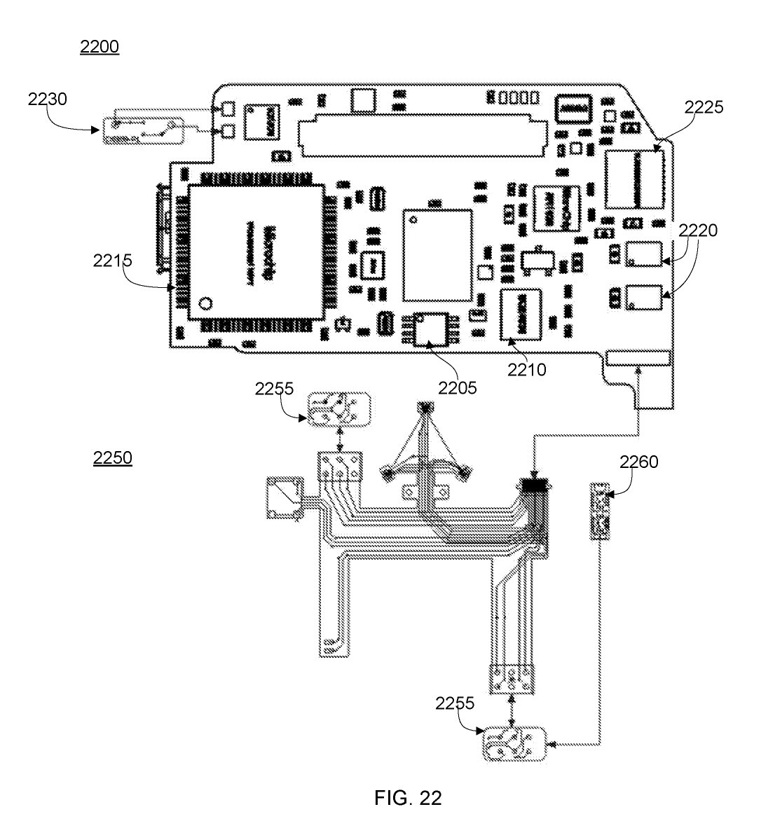

In some aspects, such as illustrated in FIG. 22, the smart cartridge 300 may comprise a processor that may communicate with the pumping mechanism 370. A flow rate sensor 315 may detect flow rate changes, detect occlusions, and sense pressure changes within the system of the smart cartridge 300, such as may be caused by air bubbles or leaks. In some embodiments, detection of an inconsistent pressure data point may prompt a disconnection from an infusion set or blockage of an outlet tube 310. In some implementations, the pumping mechanism 370 may comprise sensors that may allow for communication with one or digital valves that may be magnetically operated and controlled by the processor.

In some aspects, additional sensors may allow for detection of compressible reservoir 375, 325 medicament volumes. For example, the sensors may detect an initial fill volume and be preprogrammed with a minimum volume, wherein the processor may determine how many units may be contained within the compressible reservoir 375, 325. The sensors may transmit updated information as medicament is released. In some aspects, where the smart cartridge 300 may comprise more than one compressible reservoir 375, 325, medicament may be drawn from one compressible reservoir 375 until empty or until the minimum volume is detected and then drawn from the second compressible reservoir 325. A predefined minimum volume may limit reduction in predictability due to excessively low levels. In some aspects, the pumping mechanism 370 may alternately draw from each compressible reservoir 375, 325.

In some aspects, the smart cartridge 300 may continuously track and store information, such as the volume of medicament within the compressible reservoirs 375, 325, flow rates of distributed medicament, duration of medicament storage, internal temperature of one or more of the housing 385, 335, compressible reservoirs 375, 325, or the medicament. Continuous or periodic tracking may limit the chance of releasing contaminated medicament, which may be caused by high or low temperatures, duration without refrigeration, exposure to contaminants, as non-limiting examples. In some aspects, where the smart cartridge 300 may comprise more than one compressible reservoir 375, 325, conditions for each compressible reservoir 375, 325 may be monitored and tracked individually.

As an illustrative example, one compressible reservoir 375 may be filled to two-thirds capacity on Monday with medicament just removed from a refrigerator, and the second compressible reservoir 325 may be filled to capacity on Tuesday with medicament that had been sitting at room temperature for an hour. The medicament in the first compressible reservoir 375 may be monitored separately than the medicament in the second compressible reservoir 325. The medicament in the first compressible reservoir 375 may potentially last for two days but may be exposed to freezing temperatures on Monday night, wherein the remaining medicament may be compromised. The medicament in the second compressible reservoir 325 may potentially last for three days but may be punctured when the smart cartridge 300 is dropped on the second day.

Monitoring the compressible reservoirs 375, 325 separately may prevent the pumping mechanism 370 from mixing new medicament from the second compressible reservoir 325 with the compromised medicament from the first compressible reservoir 375 and from delivering the contaminated medicament from the second compressible reservoir 325. In some aspects, a medicament status may be continuously or periodically stored in the smart cartridge 300 processor and may be transmitted to an external device, such as the PDU connected with the smart cartridge 300, wirelessly to a smartphone, or uploaded to a healthcare provider database, wherein a healthcare provider may monitor delivery, quality, and effectiveness of the medicament over time.

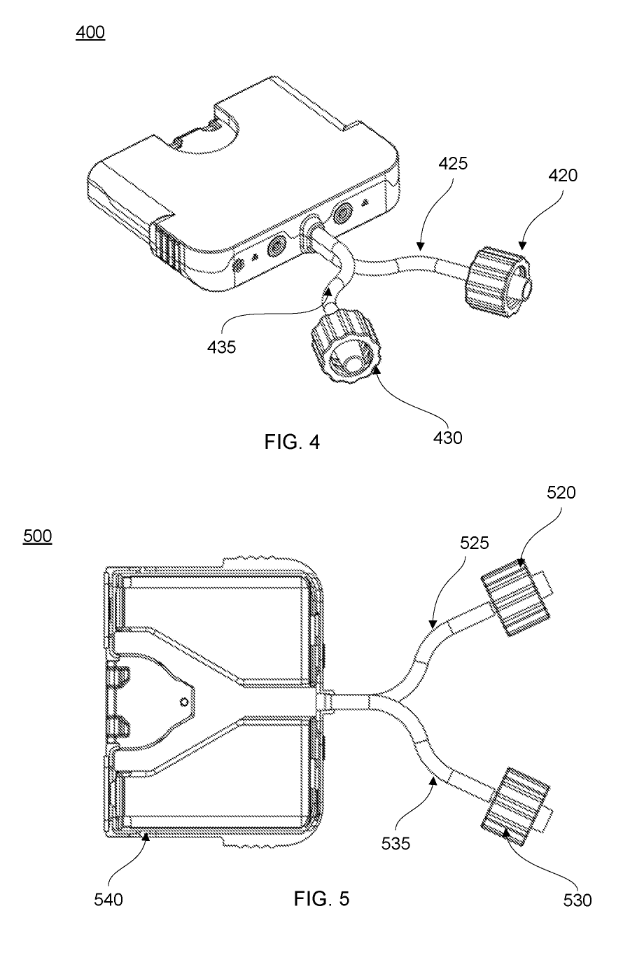

Referring now to FIG. 4, a perspective view of an alternate exemplary embodiment of a smart cartridge 400 is illustrated, wherein the smart cartridge 400 comprises two outlet tubes 425, 435, each with a separate Luer-Lock fitting 420, 430, respectively. In some aspects, the two outlet tubes 425, 435 may extend from the smart cartridge in an overlapping arrangement to limit tangling of the outlet tubes 425, 435. In some embodiments (not shown), the two outlet tubes 425, 435 may extend from different locations on the smart cartridge 400, wherein the different locations may allow for easy attachment of the Luer-Lock fittings 420, 430 to infusion sets that may be worn on separate locations of the body, such as a leg and a torso.

Referring now to FIG. 5, a top-down view of an alternate exemplary embodiment of a smart cartridge 500 is illustrated with lower housing 540, wherein the smart cartridge 500 comprises two outlet tubes 525, 535, each with a separate Luer-Lock fitting 520, 530, respectively. In some aspects, the components within the smart cartridge 500 may be organized and arranged to be overlapping, wherein the arrangement may utilize a similar amount of space as a configuration with a single outlet tube. In some embodiments, the same lower housing 540 may be used for single and dual outlet configurations, which may allow for efficient manufacturing.

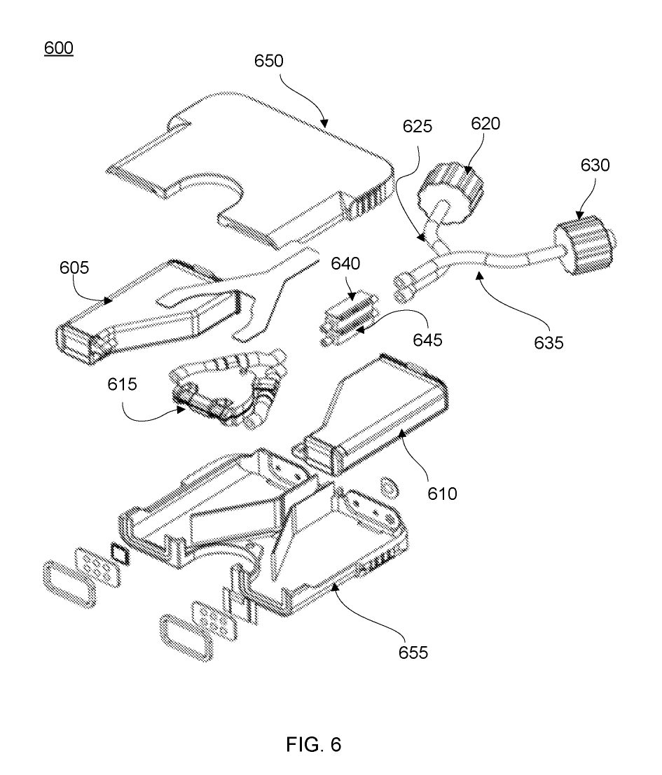

Referring now to FIG. 6, an exploded view of an alternate exemplary embodiment of a smart cartridge 600 is illustrated, wherein the smart cartridge 600 with two compressible reservoirs 605, 610 comprises two outlet tubes 625, 635, each with a separate Luer-Lock fitting 620, 630, respectively. In some aspects, the smart cartridge 600 may comprise a pumping mechanism 615 that may individually actuate each compressible reservoir 605, 610, which may allow the pumping mechanism 615 to draw different amounts from the compressible reservoirs 605, 610 and at different times and rates.

The compressible reservoirs 605, 610 may comprise the same or different medicaments and operate through distinct outlet tubes 625, 635, wherein separate flow rate sensors 640, 645 may be used to monitor the flow rates from the different compressible reservoirs 605, 610. In some embodiments, the outlet tubes 625, 635 and flow rate sensors 640, 645 may be organized as overlapping and extending from a central location between the upper housing 650 and the lower housing 655. Such organization may allow for an efficient use of space within the smart cartridge.

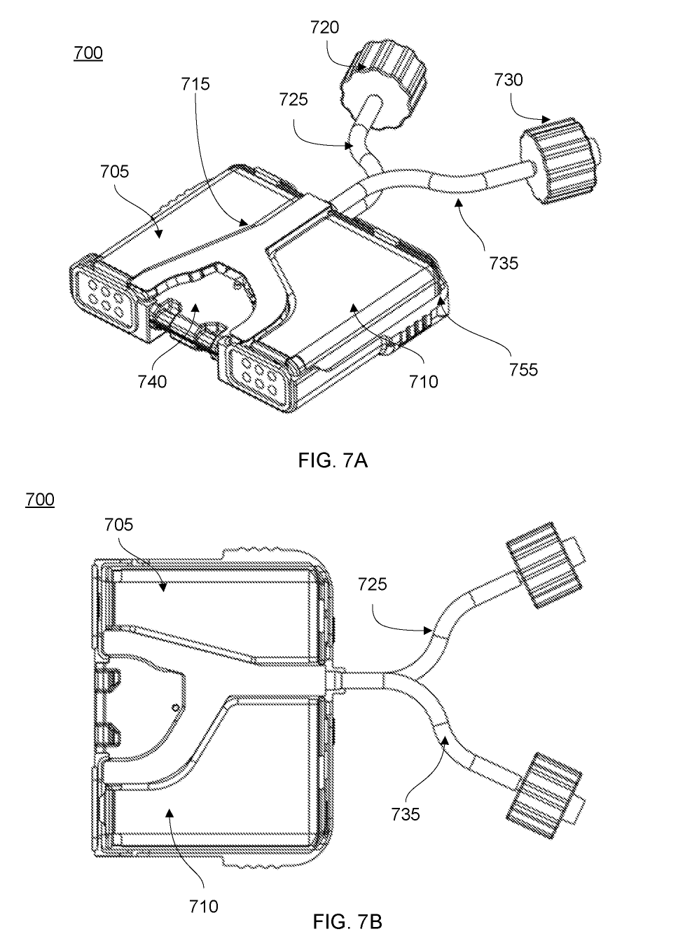

Referring now to FIG. 7A, a perspective view of an alternate exemplary embodiment of a smart cartridge 700 with lower housing 755 is illustrated, wherein the smart cartridge 700 comprises reservoirs 705, 710 with different volume capacities. Referring now to FIG. 7B, a top-down view of an alternate exemplary embodiment of a smart cartridge 700 with lower housing 755 is illustrated, wherein the smart cartridge 700 comprises reservoirs 705, 710 with different volume capacities. In some aspects, the smart cartridge 700 may comprise two outlet tubes 725, 735, each with a separate Luer-Lock fitting 720, 730, respectively. Where the compressible reservoirs 705, 710 may comprise different shapes and/or different volume capacities, the internal infrastructure 715 may be asymmetrical, wherein flow rate sensors and outlet tubes 725, 735 may be off centered within the lower housing 755.

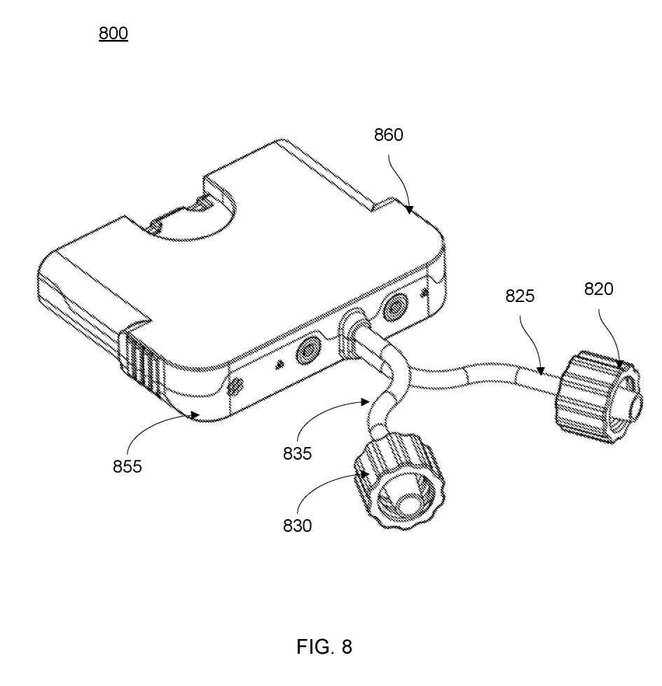

Referring now to FIG. 8, a perspective view of an alternate exemplary embodiment of a smart cartridge 800 with upper housing 860 and lower housing 855 is illustrated, wherein the smart cartridge 800 comprises reservoirs with different volume capacities. In some aspects, the smart cartridge 800 may comprise two outlet tubes 825, 835, each with a separate Luer-Lock fitting 820, 830, respectively, wherein each of the outlet tubes 825, 835 may extend from separate reservoirs within the smart cartridge 800. Where the compressible reservoirs may comprise different shapes and/or different volume capacities, the housing 855, 860 may be asymmetrical, wherein the outlet tubes 825, 835 may be held off-center within the smart cartridge 800.

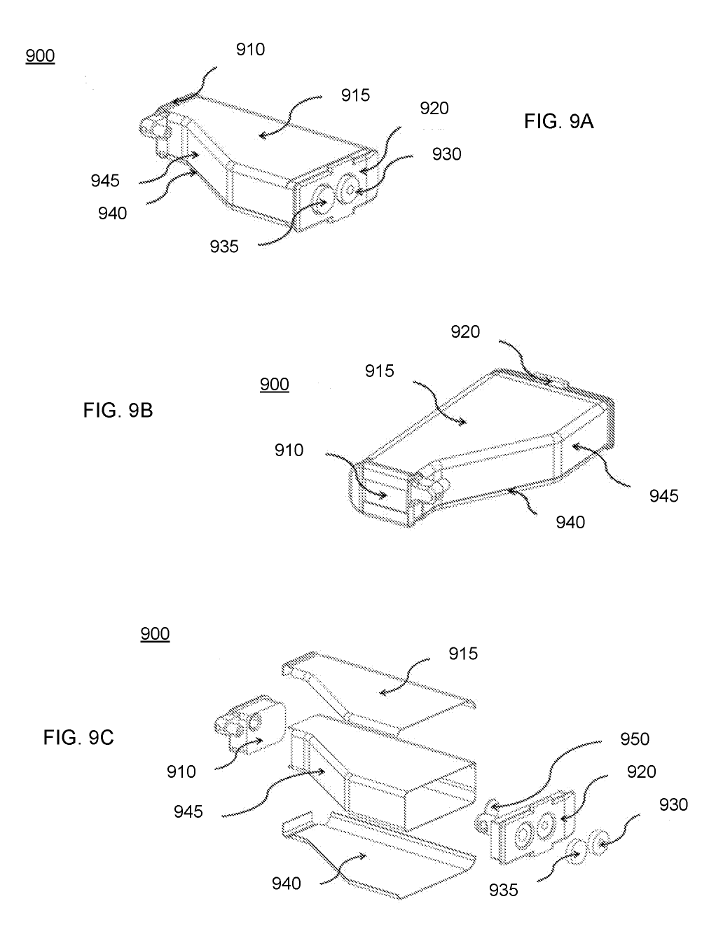

Referring now to FIGS. 9A-9C, perspective and exploded views of an exemplary embodiment of a compressible reservoir 900 for use within a dual-reservoir smart cartridge are illustrated. In some aspects, a compressible reservoir 900 may comprise a flexible pouch 945 with two end caps 910, 920. A first end cap 910 may accept tubing, wherein a pumping mechanism may draw medicament from the compressible reservoir 900 through tubing connected to the first end cap 910. A second end cap 920 may comprise a fill port 935 and an overflow port 930, wherein the fill port 935 may comprise a self-sealing septum with fill guide 950 and the overflow port 930 may comprise a pre-pierced overflow membrane. In some embodiments, the flexible pouch 945 may comprise a thin material, such as silastic or medical grade polyisoprene, as non-limiting examples.

In some aspects, the flexible pouch 945 may comprise external lining 915, 940 of a conductive material, such as aluminum, wherein the external lining 915, 940 may add rigidity to the flexible pouch 945 and conductivity between the external lining 915, 940. The external lining 915, 940 may maintain a flat surface of the flexible pouch 945 and may allow for more precise control of the compressible reservoir 900. The capacitance between the external lining 915, 940 may be directly related to the area of the external lining 915, 940 and the distance between them. Accordingly, each value of capacitance corresponds to a unique value of volume of fluid available within the compressible reservoir. In some aspects, capacitance sensing may be programmed not to exceed a maximum distance separating the external lining 915, 940, beyond which the noise floor of the sensor system may become greater than the measured capacitance itself.

In some embodiments, the external lining 915, 940 may be attached to the flexible pouch 945 through a range of adhesion processes, such as through use of an adhesive, like glue, epoxy, welding, or chemical adhesion. In some implementations, the external lining 915, 940 may coat the flexible pouch 945, wherein the external lining 915, 940 may be added in a liquid form, which may harden or dry, such as through polymerization or exposure to a threshold temperature.

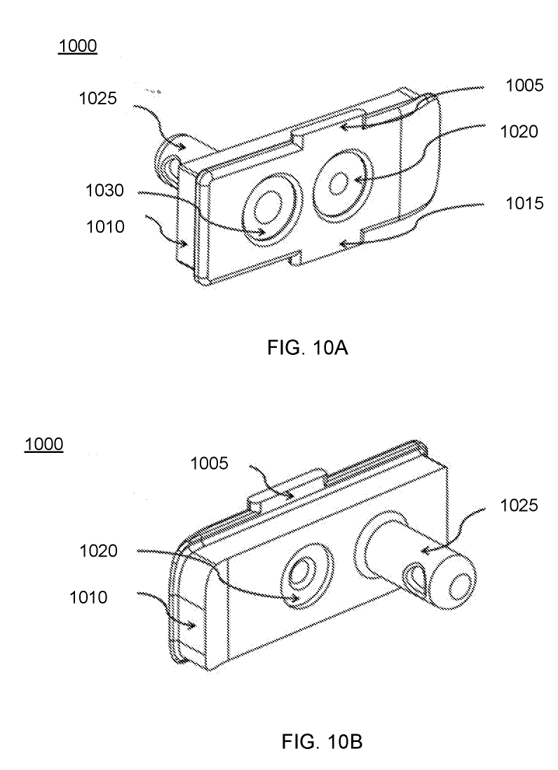

Referring now to FIGS. 10A and 10B, perspective views of an end cap 1000 for a compressible reservoir are illustrated, wherein the end cap 1000 may comprise a fill port 1030 with fill guide 1025 and an overflow port 1020. In some embodiments, the end cap 1000 may comprise fitting protrusions 1005, 1015, which may secure the end cap 1000 on or between one or both the upper housing and lower housing of a smart cartridge. In some aspects, the end cap 1000 may comprise a pouch ledge 1010, wherein the flexible pouch may be adhered to the end cap 1000.

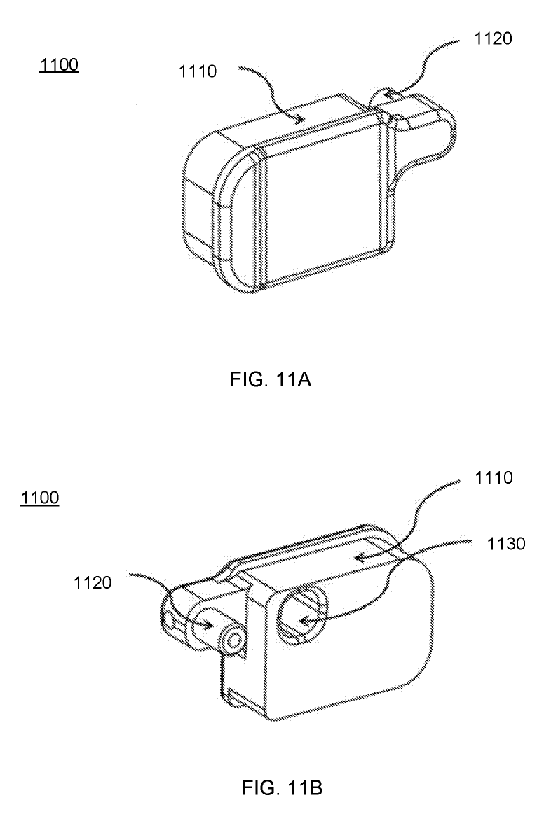

Referring now to FIGS. 11A and 11B, perspective views of an end cap 1100 for a compressible reservoir are illustrated, wherein the end cap 1100 may comprise an angled channel 1110 and a draw opening 1120 to which flexible tubing may be attached. In some aspects, the pumping mechanism of the smart cartridge may draw medicament through the flexible tubing attached to the draw opening 1120 from the compressible reservoir through an internal opening 1130. In some implementations, the medicament may flow into the angled channel 1110 from the internal opening 1130.

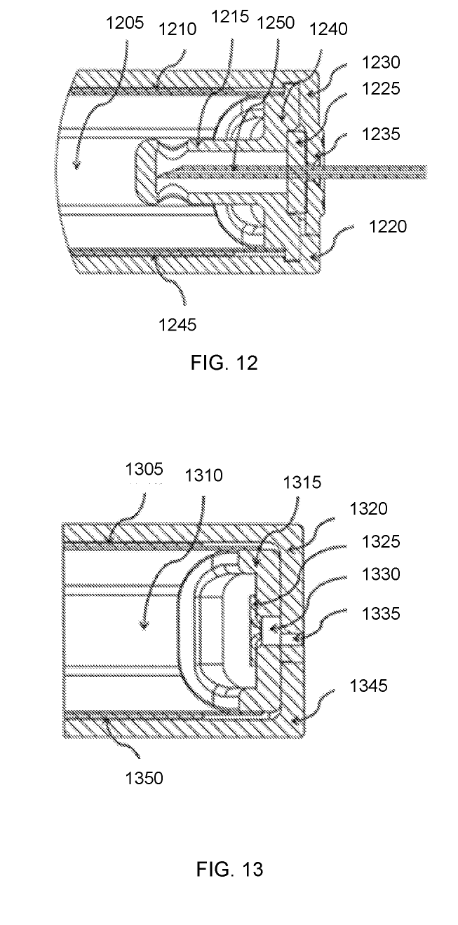

Referring now to FIG. 12, a cross section of an exemplary embodiment of a fill port 1235 for use with an end cap 1240 of a compressible reservoir 1230 of smart cartridge is illustrated. In some aspects, the fill port 1235 may comprise a membrane 1225 and a fill guide 1215 that may extend into a flexible pouch 1205, wherein the flexible pouch 1205 may comprise an external conductive lining 1210, 1245. In some embodiments, the capacitance between the external conductive lining 1210, 1245 may be monitored, wherein a user may receive an indication of how much medicament has been filled, such as through a pump display unit or other wireless communication device. In some aspects, the fill port 1235 may be located on an exterior edge of the housing 1220, wherein at least a portion of the fill port 1235 may be accessible externally.

In some aspects, the membrane 1225 may be pierced with a filling mechanism 1250, such as a syringe, and self-seal once the filling mechanism 1250 is removed. In some embodiments, the fill port 1235 may allow for filling and refilling of the compressible reservoir, wherein the self-sealing may occur multiple times. In some implementations, the fill port 1235 may comprise a unique fitting configured to accept pre-defined filling mechanisms 1250. For example, as illustrated, the fill guide 1215 may be configured to accept a male plug of a syringe. In some embodiments, the fill port 1235 may comprise an electronically controlled valve coupled to a sensor (not shown), wherein the sensor may detect the filling mechanism 1250 and the valve may allow or prohibit the fill pending authenticating the filling mechanism 1250.

As an illustrative example, a smart cartridge may be configured and prescribed specifically for use with a particular brand of insulin. The filling mechanism may be prefilled with the correct insulin and associated with a unique identification number (UIN) that may be detectable by the sensor in the fill port 1235, such as through near field communication or other low-power communication protocols.

Referring now to FIG. 13, a cross-section of an exemplary open embodiment of an overflow port 1335 for use with a smart cartridge is illustrated, wherein the overflow port 1335 may comprise an overflow membrane 1325 and a through hole 1330, which may allow excess medicament to flow out of the flexible pouch 1310 if a predefined threshold internal pressure is exceeded. In some aspects, the predefined threshold pressure may be passively sensed, wherein the properties of the overflow membrane 1325 may cause the overflow membrane 1325 to open at the predefined threshold pressure.

In some implementations, the capacitance of the external lining 1305, 1350 may be monitored, wherein the overflow membrane 1325 may be actively opened when a threshold capacitance has been reached. In some aspects, the overflow port 1335 may be located on an end cap 1315 of a compressible reservoir 1320, wherein the through hole 1330 may extend to the perimeter of the housing 1345, allowing excess medicament to flow outside of the housing 1345.

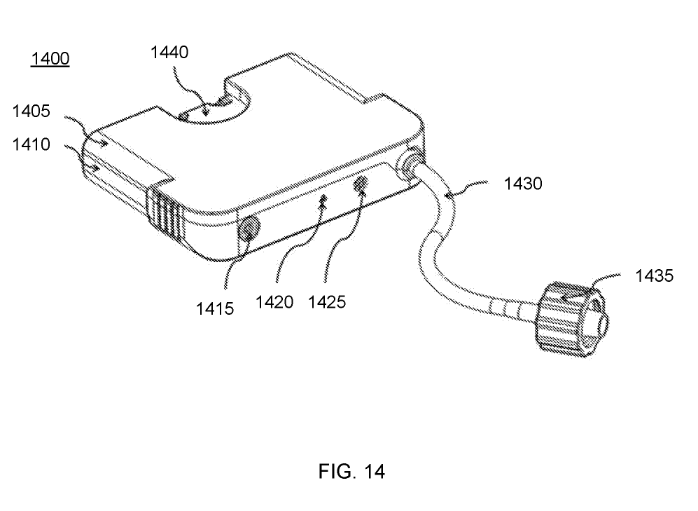

Referring now to FIG. 14, a perspective view of an exemplary smart cartridge 1400 with a single compressible reservoir is illustrated. In some aspects, a smart cartridge 1400 with a a pumping mechanism 1440 and a single compressible reservoir may comprise similar features to a smart cartridge with dual compressible reservoirs, such as illustrated in FIGS. 1A-1C. In some embodiments, the smart cartridge 1400 may comprise a fill port 1415 and an overflow port 1420. The components of the smart cartridge 1400 may be contained within an upper housing 1405 and a lower housing 1410. In some aspects, one or both the upper housing 1405 and the lower housing 1410 may comprise an opening for an outlet tube 1430 with a male Luer-Lock fitting 1435 and through holes 1425, which may reduce collection of moisture or build-up of gas pressure.

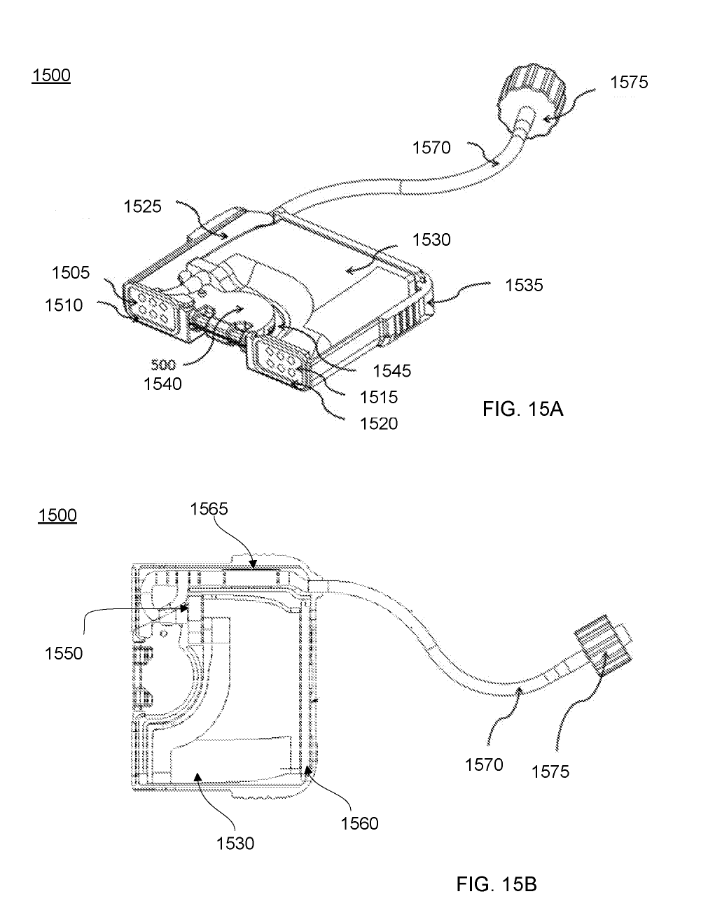

Referring now to FIGS. 15A and 15B, a perspective view and a top down view of an exemplary embodiment of a smart cartridge 1500 with a single compressible reservoir 1530 is illustrated with the lower housing 1535 and internal infrastructure 1525, which may organize, secure, and protect internal components. In some aspects, a smart cartridge 1500 with a pumping mechanism 1540 and a single compressible reservoir may comprise similar features to a smart cartridge with dual compressible reservoirs, such as illustrated in FIGS. 2A-2B.

In some implementations, the smart cartridge 1500 may comprise a plurality of target connectors 1505, 1515 with a plurality of sealing gaskets 1510, 1520, which may secure placement within a pump display unit (not shown). The target connectors 1505, 1515 and sealing gaskets 1510, 1520 may ensure alignment and connection with electrical connectors and spring-loaded pins of a pump display unit, such as illustrated in FIG. 20.

In some embodiments, the smart cartridge 1500 may comprise a first end cap 1560, which may allow for the filling of the compressible reservoir 1530, and a pumping mechanism 1540 that may draw medicament from the compressible reservoir 1530 through tubing 1545 that may be connected to a second end cap 1550. The medicament may be pushed through a flow rate sensor 1565 and then through an outlet tube 1570 with a male Luer-Lock fitting 1575, which may be attached to a dispensing device, such as an infusion set.

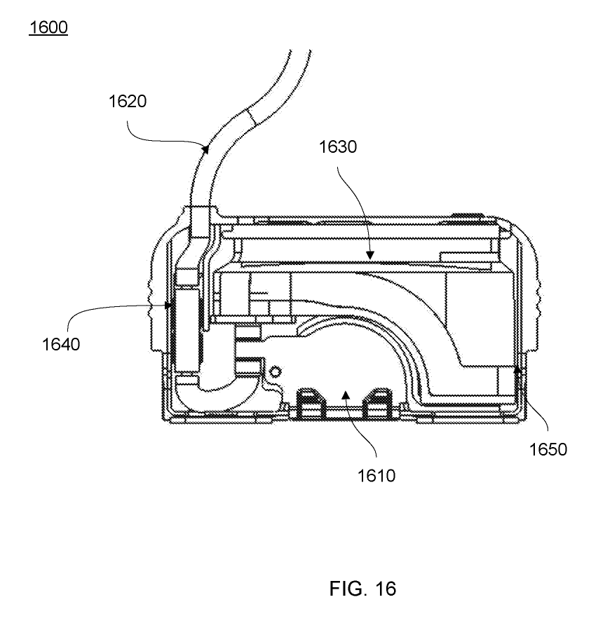

Referring now to FIG. 16, an exemplary embodiment of a smart cartridge 1600 is illustrated, wherein the threshold volume capacity of the compressible reservoir 1630 is lower than illustrated in prior figures. In some aspects, a smart cartridge 1600 with a pumping mechanism 1610 and a small, single compressible reservoir 1630 may comprise similar features to a smart cartridge with a larger, single compressible reservoir, such as illustrated in FIGS. 15A-15B. Similar to a larger, single compressible reservoir, the smart cartridge 1600 may comprise a housing 1650 that may contain the components of the smart cartridge 1600, including, for example, a flow rate sensor 1640, small compressible reservoir 1630, pumping mechanism 1610, and at least part of an outlet tube 1620.

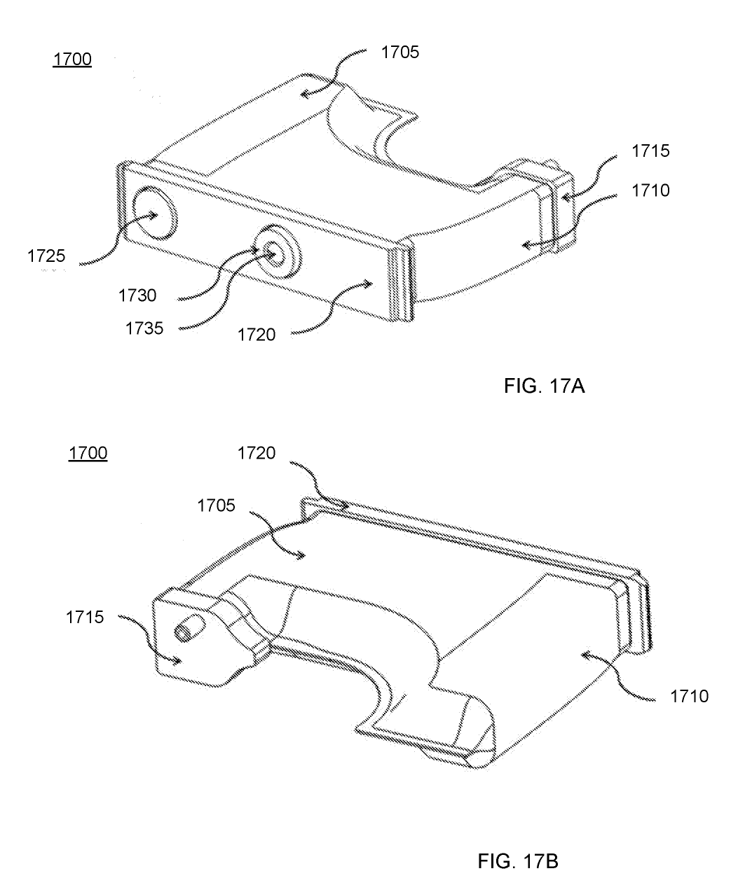

Referring now to FIGS. 17A and 17B, perspective views of a single compressible reservoir 1700 are illustrated, wherein the single compressible reservoir 1700 comprises a flexible pouch 1710, a first end cap 1715 with flow port, and a second end cap 1720 with fill port 1725 and overflow port 1730, 1735. In some aspects, the single compressible reservoir 1700 may comprise conductive external lining 1705 adhered to adjacent walls of the flexible pouch 1710, wherein a predefined distance range between the conductive external lining 1705 is associated with a predefined capacitance range. In some aspects, each capacitance within the predefined capacitance range is associated with a particular quantity of medicament contained in the single compressible reservoir 1700.

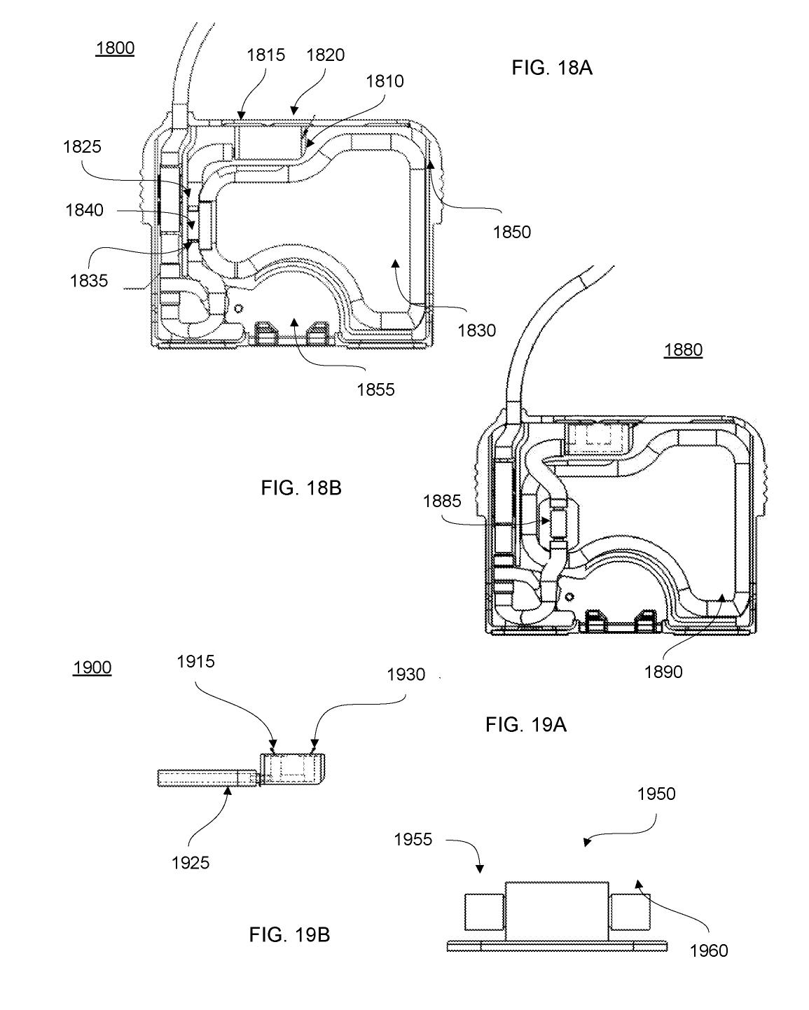

Referring now to FIG. 18A, an alternate exemplary embodiment of a smart cartridge 1800 with a single compressible reservoir 1830 is illustrated, wherein the single compressible reservoir 1830 comprises a dual port end cap 1840. In some aspects, a dual port end cap 1840 may allow for a larger threshold volume capacity of the compressible reservoir 1830 in a similar sized housing 1850 as a compressible reservoir with two end caps, such as illustrated in FIG. 15. The dual port end cap 1840 may comprise an intake port 1825 and a flow port 1835. In some aspects, a pumping mechanism 1855 may draw medicament from the compressible reservoir 1830 through internal tubing connected to the flow port 1835. In some embodiments, the intake port 1825 may be connected to a fill fitting 1810, which may comprise a fill port 1820 and an overflow port 1815.

Referring now to FIG. 18B, an alternate exemplary embodiment of a smart cartridge 1880 with a single compressible reservoir 1890 is illustrated, wherein the single compressible reservoir 1890 comprises a dual port fitting 1885. In some implementations, the dual port fitting 1885 may be located on the front of the compressible reservoir 1890, which may efficiently share space within the smart cartridge 1880. The efficient use of space may allow for a compressible reservoir 1890 with a higher volume capacity.

Referring now to FIGS. 19A and 19B, an exemplary fill fitting 1900 and a dual port end cap 1950 are illustrated, wherein the fill fitting 1900 and the dual port 1950 may be used in conjunction with a compressible reservoir of a smart cartridge. In some aspects, the fill fitting 1900 may comprise a fill port 1930 and an overflow port 1915, wherein medicament received through the fill port 1930 may flow through the same flexible tubing 1925 as overflow medicament expelled through the overflow port 1915. In some embodiments, the dual port end cap 1950 may be heat-sealed to the flexible pouch. In some implementations, the dual port end cap 1950 may comprise an intake port 1960 and a flow port 1955, wherein the intake port 1960 may receive medicament from the flexible tubing 1925 of the fill fitting 1900.

Referring now to FIGS. 20A-20B, an exemplary embodiment of a pumping mechanism 2000 engaged with an external control unit is illustrated, wherein control unit electromagnets 2035, 2040 may control a stroke of pump magnets 2005, 2010 interacting through a membrane 2015. In some aspects, the external control unit may comprise a locking mechanism 2055 with a spring 2045 and clamping 2070, 2075 that may engage with cartridge grooves 2060, 2065. In some embodiments, the control unit may comprise a plurality of triaxial Hall effect sensors 2020-2030.

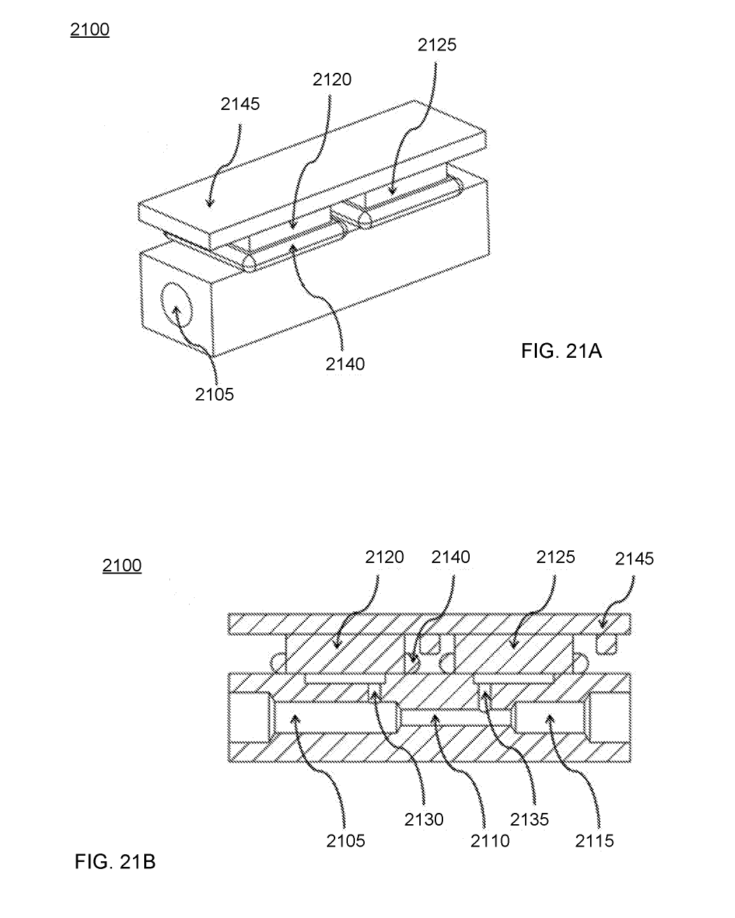

Referring now to FIGS. 21A-21B, an exemplary embodiment of a flow rate sensor 2100 is illustrated, wherein the flow rate sensor 2100 may detect flow of a medicament from a compressible reservoir through an outlet tube. In some aspects, the flow rate sensor 2100 may measure and calculate a dual flow rate. In some embodiments, a fluid at original pressure may be pushed through a first channel 2105, through a second channel 2110 reaching a second pressure, and finally through a third channel 2115. In some aspects, the second channel 2110 may have a diameter less than the first channel 2105. For example, the first channel 2105 may have a diameter of 0.031 inch and the second channel 2110 may have a diameter of 0.015 inch.

In some implementations, the flow rate sensor 2100 may comprise one or more pressure sensors 2120, 2125, wherein the pressure sensors 2120, 2125 may measure the drop in pressure between the first channel 2105 and the second channel 2110. The Venturi effect may allow for the calculation of the volumetric flow rate based on the first measured pressure by the first fluid pressure sensor 2120 through a first measurement channel 2130 and the second measured pressure by the second fluid pressure sensor 2125 through a second measurement channel 2135.

In some aspects, pressure sensors 2120, 2125 may be hermetically bonded, such as through use of bonded joints 2140, to the body of the flow rate sensor 2100. In some embodiments, the measurement channels 2130, 2135 may be filled with a biocompatible gel that may insulate the pressure sensors 2120, 2125 from the fluid, wherein the insulation may increase the sensitivity of the flow rate sensor 2100, which may enhance its accuracy. In some aspects, a printed circuit board 2145 may be mounted and attached onto the pressure sensors 2125, 2120, which may allow for communication between the flow rate sensor 2100 and a microcontroller unit, such as illustrated in FIG. 3.

Referring now to FIG. 22, exemplary embodiments of wireless circuit boards of a pump display unit (PDU) 2200 and a smart cartridge 2250 are illustrated, wherein the smart cartridge 2250 may be inserted into the PDU 2200 allowing for electrical communication between the smart cartridge 2250 and the PDU 2200. In some embodiments, the PDU 2200 may comprise a battery printed circuit board (“PCB”) 2230, which may control the power of one or both the PDU 2200 or the smart cartridge 2250. In some aspects, the PDU 2200 may comprise a microcontroller 2215, an audio digital to analog converter 2225, real time clock 2205, and a charge control 2210. In some embodiments, the PDU 2200 may comprise one or more pump drivers 2220, wherein the pump drivers 2220 may control at least some of the functionality of the pump mechanism on the smart cartridge 2250.

In some aspects, the smart cartridge 2250 may comprise a flow sensor PCB 2260, which may interface with the flow rate sensor. The smart cartridge 2250 may comprise one or more cartridge PCBs 2255, which may control and process data from the smart cartridge 2250. In some implementations, each cartridge PCB 2255 may manage and control individual reservoirs, such as illustrated in FIG. 3.

In some aspects, the PDU 2200 and the smart cartridge 2250 may share the processing and control of the components of one or both the PDU 2200 and the smart cartridge 2250. For example, a battery PCB 2230 of the PDU 2200 may provide power to the smart cartridge 2250, allowing the flow sensor PCB 2260 to process the flow rate sensor data.

In some embodiments, the smart cartridge 2250 may process a number of calculations and then share the processed data with the PDU 2200. In some aspects, the smart cartridge 2250 may comprise a sensor interface that may detect one or both the number of reservoirs and the capacities of each reservoir, which may allow for interchangeability between smart cartridges 2250 with different reservoir quantities and volume capacities. Such flexibility on the smart cartridge 2250 may reduce the need for different PDUs 2200 as the smart cartridge 2250 may transmit specifications to the PDU 2200, allowing for informed control of the pumping mechanism.

In some aspects, the smart cartridge 2250 may be filled and/or refilled with a variety of subcutaneous drugs. Once inserted into the PDU 2200, the smart cartridge 2250 may deliver medicaments such as insulin, which may include long or slow acting options to lower the patient's blood glucose level, amylin analogues such as Pramlintide, or glucagon to raise the patient's blood glucose level. As another illustrative example, the smart cartridge 2250 may deliver subcutaneous medication for pain management. The subcutaneous infusion of a range of liquid medications may be possible with the combination of the PDU 2200 and a smart cartridge 2250 inserted into the PDU.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A smart cartridge, comprising:
a first compressible reservoir comprising:
a first fill port configured to accept a filling mechanism, wherein the filling mechanism is configured to add a first medicament to the first compressible reservoir;
a first overflow port configured to dispel an excess amount of the first medicament where the first medicament filled exceeds a first threshold volume capacity within the first compressible reservoir;
a first flow port through which the first medicament flows for use;
a first flexible pouch configured to contain the first medicament;
a first internal tubing connected to the first flow port;
a pumping mechanism operably connected to the first internal tubing and configured to be electrically connected to an external power source, wherein the pumping mechanism controls flow of the first medicament from the first compressible reservoir;
a flow rate sensor configured to monitor a flow of the first medicament from the first internal tubing to the first outlet tube;
a first outlet tube connected to the first internal tubing through which a first expulsion of the first medicament to an external body flows; and
a housing containing the first compressible reservoir, the first internal tubing, the pumping mechanism, the flow rate sensor, and at least part of the first outlet tube.

2. The smart cartridge of claim 1, wherein the housing comprises gas permeable through ports.

3. The smart cartridge of claim 1, wherein the first compressible reservoir further comprises a first end cap with the first fill port, the first overflow port, and the first flow port.

4. The smart cartridge of claim 1 further comprises a first volume sensor configured to recognize a volume level of the first medicament contained within the first compressible reservoir.

5. The smart cartridge of claim 1, wherein the first compressible reservoir further comprises:
a first end cap with the first fill port and the first overflow port; and
a second end cap with the first flow port.

6. The smart cartridge of claim 1, wherein the first flow port comprises a first internal tubing fitting mechanism that secures a connection between the first internal tubing and the first flow port.

7. The smart cartridge of claim 1, wherein the first compressible reservoir further comprises:
a first conductive layer adhered to a first wall of the first flexible pouch; and
a second conductive layer adhered to a second wall of the first flexible pouch proximate to the second wall, wherein a predefined distance range between the first conductive layer and the second conductive layer is associated with a predefined capacitance range, and wherein each capacitance within the predefined capacitance range is associated with a first quantity of the first medicament contained in the first compressible reservoir.

8. The smart cartridge of claim 1, further comprising a second compressible reservoir comprising:
a second fill port configured to accept a second filling mechanism, wherein the second filling mechanism is configured to add a second medicament to the second compressible reservoir;
a second overflow port configured to dispel an excess amount of the second medicament where the second medicament filled exceeds a second threshold volume capacity within the second compressible reservoir;
a second flow port through which the second medicament flows for use; and
a second flexible pouch configured to contain the second medicament.

9. The smart cartridge of claim 8, wherein a second expulsion of the second medicament from the second compressible reservoir occurs through the first outlet tube.

10. The smart cartridge of claim 9, wherein the pumping mechanism alternately draws from the first compressible reservoir and the second compressible reservoir.

11. The smart cartridge of claim 9, wherein the pumping mechanism controls flow of the first medicament from the first compressible reservoir until the first compressible reservoir reaches a minimum medicament volume threshold before controlling flow from the second compressible reservoir.

12. The smart cartridge of claim 8, further comprising a second outlet tube, wherein a second expulsion of the second medicament from the second compressible reservoir occurs through the second outlet tube.

13. The smart cartridge of claim 12, wherein the pumping mechanism alternately draws from the first compressible reservoir and the second compressible reservoir.

14. The smart cartridge of claim 12, wherein the pumping mechanism controls flow of the first medicament from the first compressible reservoir until the first compressible reservoir reaches a minimum medicament volume threshold before controlling flow from the second compressible reservoir.

15. The smart cartridge of claim 8, wherein the first medicament and the second medicament are the same.

16. The smart cartridge of claim 8, wherein the first medicament and the second medicament are different.

17. The smart cartridge of claim 8, wherein the first threshold volume capacity and the second threshold volume capacity are the same.

18. The smart cartridge of claim 8, wherein the first threshold volume capacity is different from the second threshold volume capacity.

19. The smart cartridge of claim 1, wherein the first compressible reservoir is refillable.

20. The smart cartridge of claim 1, wherein the smart cartridge is configured to be functional for one fill of the first medicament.
